(19) United States
(12) Patent Application Publication
(10) Pub. No.: US 2008/0154282 A1
(43) Pub. Date: Jun. 26, 2008

(54) Fluidic Coupling For Surgical Hand Piece

(76) Inventors: Stacy Faught, Aliso Viejo, CA (US); Glenn Sussman, Laguna Nigel, CA (US)

Correspondence Address: ALCON, IP LEGAL, TB4-8, 6201 SOUTH FREEWAY, FORT WORTH, TX, 76134, US

(21) Appl. No.: 11/953,329

(22) Filed: Dec. 10, 2007

Related U.S. Application Data
(60) Provisional application No. 60/870,938, filed on Dec. 20, 2006.

(51) Int. Cl. A61F 9/00; F16L 39/00

(52) U.S. Cl. 606/107; 285/124.2

(57) ABSTRACT

A fluidic coupling for a surgical hand piece includes first and second sealing plates, a ring portion, and a receiving portion. The first sealing plate has a first channel for receiving a fluid. The second sealing plate has a second channel for receiving a fluid. The ring portion protrudes from the first sealing plate around a periphery of the first channel. The receiving portion extends from the second sealing plate around a periphery of the second channel. The receiving portion is movable in an axial direction along the second channel and is biased outward from a surface of the second sealing plate. When the ring portion is pressed against the receiving portion, the receiving portion provides a sealing force against the ring portion such that the first and second channels provide a continuous leak-resistant path for a fluid.

### FIELD OF THE INVENTION

The present invention relates to a fluidic coupling for a surgical hand piece and more particularly to coupling that fluidly connects a hand piece base to a hand piece tip for use in cataract surgery.

### BACKGROUND OF THE INVENTION

The human eye functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, an opening is made in the anterior capsule and a thin phacoemulsification cutting tip is inserted into the diseased lens and vibrated ultrasonically. The vibrating cutting tip liquefies or emulsifies the lens so that the lens may be aspirated out of the eye. The diseased lens, once removed, is replaced by an artificial lens.

Alcon Laboratories of Fort Worth, Tex. has developed another technology for removing cataractous lenses. This technology, known as AquaLase™, uses pulses of heated liquid, along with irrigation and aspiration, to remove the lens. These pulses are directed through a cannula that is inserted into the eye. The hand piece base supplies the pulses of heated liquid to the hand piece tip. The use of pulses of heated liquid requires a good seal between the hand piece base and the hand piece tip. Therefore, a need exists for an improved fluidic coupling for such a hand piece

### SUMMARY OF THE INVENTION

In one embodiment consistent with the principles of the present invention, the present invention is a fluidic coupling for a surgical hand piece. The fluidic coupling includes first and second sealing plates, a ring portion, and a receiving portion. The first sealing plate has a first channel for receiving a fluid. The second sealing plate has a second channel for receiving a fluid. The ring portion protrudes from the first sealing plate around a periphery of the first channel. The receiving portion extends from the second sealing plate around a periphery of the second channel. The receiving portion is movable in an axial direction along the second channel and is biased outward from a surface of the second sealing plate. When the ring portion is pressed against the receiving portion, the receiving portion provides a sealing force against the ring portion such that the first and second channels provide a continuous leak-resistant path for a fluid.

In another embodiment consistent with the principles of the present invention, the present invention is surgical device including a tip segment and a hand piece segment. The tip segment has a first sealing plate, a ring portion, and a cannula. The first sealing plate is located on a bottom surface of the tip segment. The first sealing plate has a first channel for receiving a fluid. The ring portion protrudes from the first sealing plate around a periphery of the first channel. The cannula is fluidly coupled to the first channel. The hand piece segment includes a second sealing plate with a receiving portion. The second sealing plate is located on a top surface of the hand piece segment. It has a second channel for receiving a fluid. The receiving portion extends from the second sealing plate around a periphery of the second channel. It is movable in an axial direction along the second channel and is biased outward from a surface of the second sealing plate. When the ring portion is pressed against the receiving portion, the receiving portion provides a sealing force against the ring portion such that the first and second channels provide a continuous leak-resistant path for a fluid.

In another embodiment consistent with the principles of the present invention, the present invention is an ophthalmic surgical device for providing heated pulses of a fluid for the removal of a cataractous lens. The device includes a tip segment and a hand piece segment. The tip segment has a first sealing plate, a ring portion, and a cannula. The first sealing plate is located on a bottom surface of the tip segment. The first sealing plate has a first channel for receiving a fluid. The ring portion protrudes from the first sealing plate around a periphery of the first channel. The cannula is fluidly coupled to the first channel. The cannula is adapted to be inserted into the anterior chamber of an eye and to provide a heated fluid for removal of a cataractous lens. The hand piece segment includes a second sealing plate with a receiving portion. The second sealing plate is located on a top surface of the hand piece segment. It has a second channel for receiving a fluid. The receiving portion extends from the second sealing plate around a periphery of the second channel. It is movable in an axial direction along the second channel and is biased outward from a surface of the second sealing plate. When the ring portion is pressed against the receiving portion, the receiving portion provides a sealing force against the ring portion such that the first and second channels provide a continuous leak-resistant path for a fluid.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a cross section view of a tip segment and a hand piece segment according to an embodiment of the present invention.

FIG. 2 is a view of a sealing plate located on a tip segment according to an embodiment of the present invention.

FIG. 3 is a side cross section view of a sealing plate located on a tip segment according to an embodiment of the present invention.

FIG. 4 is a side cross section view of a sealing plate located on a tip segment and a sealing plate located on a hand piece segment according to an embodiment of the present invention.

FIG. 5 is a partial side cross section view of a sealing plate located on a tip segment and a sealing plate located on a hand piece segment according to an embodiment of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

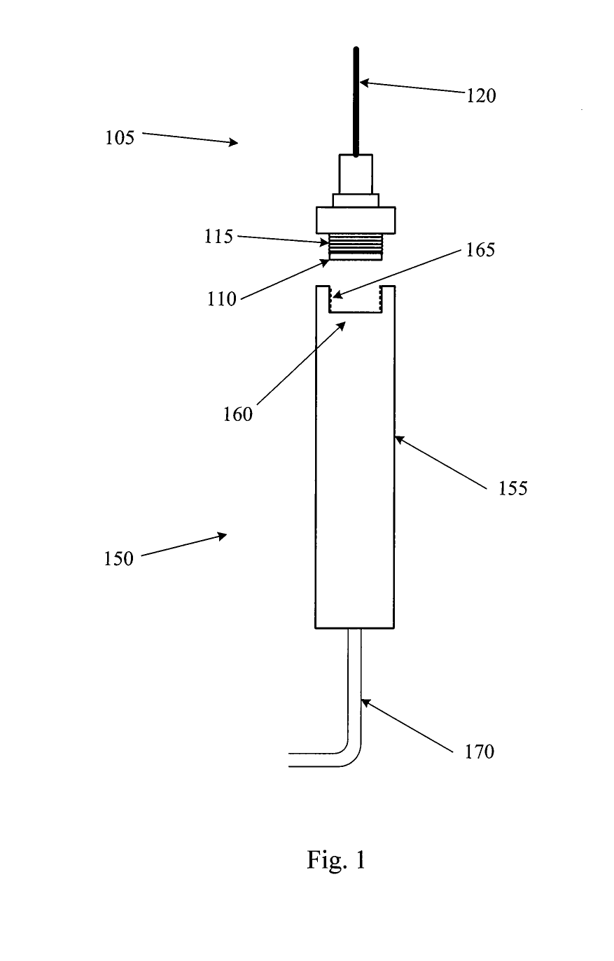

FIG. 1 is a cross section view of a tip segment and a hand piece segment according to an embodiment of the present invention. Tip segment 105 includes a tip sealing plate 110, a tip threaded portion 115, and a cannula 120. Hand piece segment 150 includes a body 155, a hand piece sealing plate 160, a hand piece threaded portion 165, and tubing 170.

In tip segment 105, tip sealing plate 110 is located on a bottom surface of tip segment 105. Tip segment sealing plate 110 is located adjacent to tip threaded portion 115. Cannula 120 is located on and extends from a top surface of tip segment 105.

In hand piece segment 150, hand piece sealing plate 160 is located near a top surface of body 155. Hand piece threaded portion 165 extends upward from body 155 to form a recess into which tip threaded portion 115 may be received. Tubing 170 extends from an end of hand piece segment 150.

In the configuration of FIG. 1, tip threaded portion 115 is designed to be engageable with and disengageable from hand piece threaded portion 165. In this manner, tip segment 105 can be connected to and disconnected from hand piece segment 150. Tip piece 105 is screwed into and unscrewed from hand piece segment 150.

When tip segment 105 is connected to hand piece 150, a continuous path for a fluid is formed from tubing 170, through body 155, through hand piece sealing plate 160, through tip sealing plate 110, through tip threaded portion 115, and through cannula 120. In this manner, fluid can be ejected from cannula 120.

Typically, cannula 120 is designed to be inserted into the anterior chamber of an eye in a procedure to remove a cataractous lens. Pulses of heated fluid are ejected from cannula 120 to erode the lens. In addition, cannula 120 is designed to provide irrigation and aspiration functions to assist in the removal of the lens.

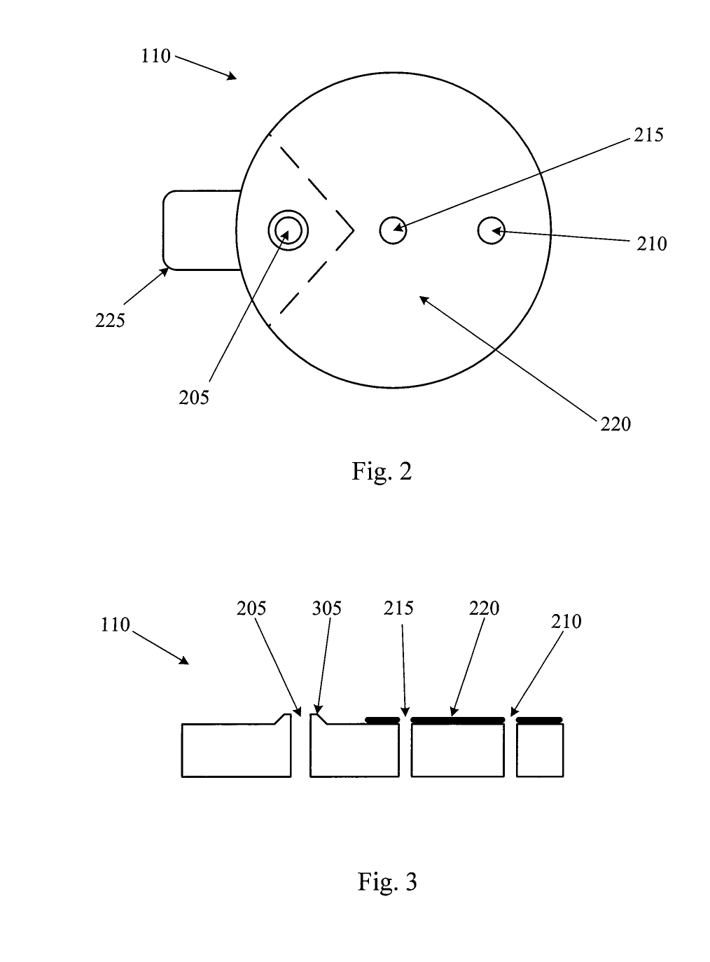

FIG. 2 is a view of a sealing plate located on a tip segment according to an embodiment of the present invention. FIG. 2 depicts the tip sealing plate 110 as viewed from the bottom of the tip segment. In FIG. 2, tip sealing plate 110 includes pulse channel 205, irrigation channel 210, aspiration channel 215, gasket 220, and tab 225.

Tab 225 extends outward along a periphery of tip sealing plate 110. Typically, tab 225 assists in aligning tip segment 110 and hand piece 150 when they are being connected together. Pulse channel 205 extends from the bottom surface into the interior of tip sealing plate 110. Like wise, irrigation channel 210 and aspiration channel 215 also extend from the bottom surface into the interior of tip sealing plate 110. Gasket 220 is located on the bottom surface of tip sealing plate 110. Gasket 220 is in the shape of a circle with a pie shaped wedge removed from it. In this manner, gasket 220 lies over the region of the bottom surface of tip sealing plate 110 that is bounded by the dashed lines and the circular perimeter and includes irrigation channel 210 and aspiration channel 215. Gasket 220 does not cover the region of the bottom surface of tip sealing plate 110 that includes pulse channel 205.

Pulse channel 205 is designed to deliver pulses of heated fluid to the cannula (not shown). Irrigation channel 210 is designed to provide irrigation through the cannula. Aspiration channel 215 is designed to provide aspiration through the cannula.

FIG. 3 is a side cross section view of a sealing plate located on a tip segment according to an embodiment of the present invention. In FIG. 3, tip sealing plate 110 includes pulse channel 205, ring portion 305, irrigation channel 210, aspiration channel 215, and gasket 220.

Ring portion 305 protrudes from the surface of sealing plate 110 and around the periphery of pulse channel 205. Pulse channel 205 typically has a circular cross section. In such a case, ring portion 305 is also circular in shape. In the embodiment of FIG. 3, ring portion 305 has a sloped portion and a flat portion. Ring portion 305 is configured to facilitate a seal for the pulse channel 205.

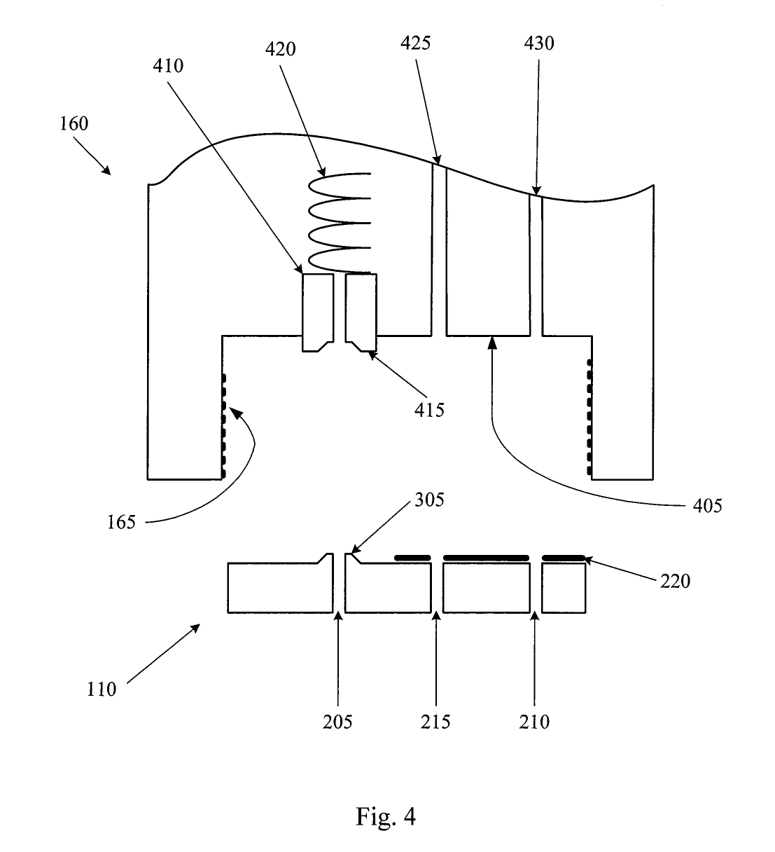

FIG. 4 is a side cross section view of a sealing plate located on a tip segment and a sealing plate located on a hand piece segment according to an embodiment of the present invention. In FIG. 4, tip sealing plate 110 includes pulse channel 205, ring portion 305, irrigation channel 210, aspiration channel 215, and gasket 220. Hand piece sealing plate 160 includes hand piece threaded portion 165, bottom surface 405, pulse connector 410, receiving portion 415, spring 420, hand piece irrigation channel 425, and hand piece aspiration channel 430.

Tip sealing plate 110 is designed to fit on the bottom surface 405 of hand piece sealing plate 160 such that irrigation channel 210 and aspiration channel 215 align with hand piece irrigation channel 425 and hand piece aspiration channel 430. When tip segment 105 is connected to hand piece 150, tip sealing plate 110 is located adjacent to bottom surface 405. Gasket 220 facilitates a seal between irrigation channel 210 and hand piece irrigation channel 425 and between aspiration channel 215 and hand piece aspiration channel 430. Ring portion 305 contacts receiving portion 415 of pulse connector 410 and pushes it upward. Spring 420 biases pulse connector 410 toward ring portion 305 and provides a sealing force that holds receiving portion 415 of pulse connector 410 against ring portion 305 and the top surface of tip sealing plate 110 surrounding ring portion 305. While spring 420 is shown, any other type of mechanism may be used to provide the force that biases pulse connector 410 toward ring portion 305 and provides the sealing force. For example, a spring washer may be used.

Pulse connector 410 is located adjacent to spring 420. Spring 420 is arranged such that it applies a spring force that pushes pulse connector 410 downward from bottom surface 405 of hand piece sealing plate 160. In this manner, pulse connector 410 protrudes from bottom surface 405 of hand piece sealing plate 160. Pulse connector 410 is designed to move axially (up and down with respect to bottom surface 405 in FIG. 4) along pulse channel 205. As noted, receiving portion 415 is designed to engage ring portion 305 and provide a seal between pulse connector 410 and ring portion 305.

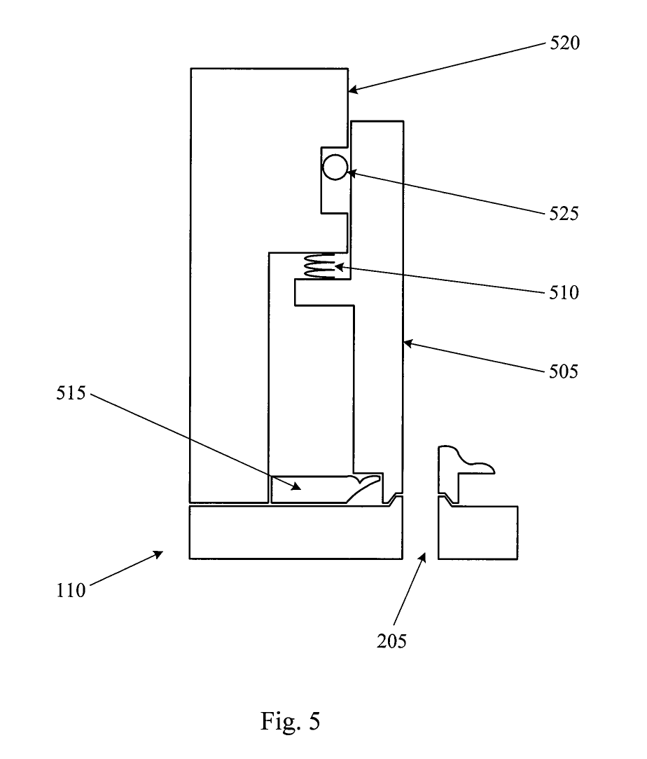

FIG. 5 is a partial side cross section view of a sealing plate located on a tip segment and a sealing plate located on a hand piece segment according to an embodiment of the present invention. In FIG. 5, pulse channel 205 is shown on a portion of tip sealing plate 110. Also depicted are a portion of pulse connector 505, spring 510, spring washer 515, a portion of hand piece sealing plate 520, and o-ring 525. FIG. 5 depicts only the portion of the apparatus that is associated with sealing pulse channel 205.

As shown in the embodiment of FIG. 5, tip sealing plate 110 is in a sealed position. The portion of pulse connector 505 is pushed upward by and is exerting a downward force on tip sealing plate 110. Spring washer 515 and spring 510 each provide a force that biases the pulse connector toward the tip sealing plate. If tip sealing plate 110 were removed, then the pulse connector would protrude downward. While both spring 510 and spring washer 515 are shown in this embodiment, in other embodiments only one of them may be present. O-ring 525 provides a seal between the portion of pulse connector 510 and the portion of the hand piece sealing plate shown in FIG. 5.

In operation, when tip sealing plate 110 is sealed against the pulse connector, pulses of heated fluid pass through pulse channel 205. The pulses of heated fluid pass through the hand piece segment 150 along a path adjacent to the portion of the pulse connector 505 shown in FIG. 5. The sealing force provided by the spring 510 and the spring washer 515 serve to create a fluid resistant seal that allows pulses of heated fluid to pass through the hand piece, through the tip segment, and out of the cannula.

From the above, it may be appreciated that the present invention provides an improved system for fluidly coupling a hand piece base to a hand piece tip. The present invention provides a sealing mechanism that prevents leakage during the cataract removal process. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A fluidic coupling for a surgical hand piece comprising:
a first sealing plate having a first channel, the first channel for receiving a fluid;
a second sealing plate having a second channel, the second channel for receiving a fluid;
a ring portion protruding from the first sealing plate around a periphery of the first channel; and
a receiving portion extending from the second sealing plate around a periphery of the second channel, the receiving portion movable in an axial direction along the second channel, the receiving portion biased outward from a surface of the second sealing plate;
wherein when the ring portion is pressed against the receiving portion, the receiving portion provides a sealing force against the ring portion such that the first and second channels provide a continuous leak-resistant path for a fluid.

2. The fluidic coupling of claim 1 further comprising:
a spring located adjacent to the receiving portion, the spring for biasing the receiving portion toward the ring portion and for providing the sealing force.

3. The fluidic coupling of claim 1 further comprising:
a spring washer located adjacent to the receiving portion, the spring washer for biasing the receiving portion toward the ring portion and for providing the sealing force.

4. The fluidic coupling of claim 1 further comprising:
a third channel located on the first sealing plate;
a fourth channel located on the second sealing plate; and
a gasket located between the first and second sealing plates.

5. The fluidic coupling of claim 4 wherein when the ring portion is pressed against the receiving portion, the third and fourth channels provide a continuous path for irrigation.

6. The fluidic coupling of claim 4 wherein when the ring portion is pressed against the receiving portion, the third and fourth channels provide a continuous path for aspiration.

7. The fluidic coupling of claim 1 further comprising:
a first threaded portion located around the periphery of the first sealing plate; and
a second threaded portion located on an interior surface of the second sealing plate and extending from a surface on which the second channel is located;
wherein the first threaded portion is engageable with the second threaded portion.

8. A surgical device comprising:
a tip segment comprising:
a first sealing plate located on a bottom surface of the tip segment, the first sealing plate having a first channel, the first channel for receiving a fluid;
a ring portion protruding from the first sealing plate around a periphery of the first channel; and
a cannula fluidly coupled to the first channel; and
a hand piece segment comprising:
a second sealing plate located on a top surface of the hand piece segment, the second sealing plate having a second channel, the second channel for receiving a fluid; and
a receiving portion extending from the second sealing plate around a periphery of the second channel, the receiving portion movable in an axial direction along the second channel, the receiving portion biased outward from a surface of the second sealing plate;
wherein when the ring portion is pressed against the receiving portion, the receiving portion provides a sealing force against the ring portion such that the first and second channels provide a continuous leak-resistant path for a fluid.

9. The fluidic coupling of claim 8 further comprising:
a spring located adjacent to the receiving portion, the spring for biasing the receiving portion toward the ring portion and for providing the sealing force.

10. The fluidic coupling of claim 8 further comprising:
a spring washer located adjacent to the receiving portion, the spring washer for biasing the receiving portion toward the ring portion and for providing the sealing force.

11. The fluidic coupling of claim 8 further comprising:
a third channel located on the first sealing plate;
a fourth channel located on the second sealing plate; and
a gasket located between the first and second sealing plates.

12. The fluidic coupling of claim 11 wherein when the ring portion is pressed against the receiving portion, the third and fourth channels provide a continuous path for irrigation.

13. The fluidic coupling of claim 11 wherein when the ring portion is pressed against the receiving portion, the third and fourth channels provide a continuous path for aspiration.

14. The fluidic coupling of claim 8 further comprising:
a first threaded portion located on the tip segment around the periphery of the first sealing plate; and
a second threaded portion located on the hand piece segment on an interior surface of the second sealing plate and extending from a surface on which the second channel is located;
wherein the first threaded portion is engageable with the second threaded portion.

15. An ophthalmic surgical device for providing heated pulses of a fluid for the removal of a cataractous lens, the device comprising:
a tip segment comprising:
a first sealing plate located on a bottom surface of the tip segment, the first sealing plate having a first channel, the first channel for receiving a fluid;
a ring portion protruding from the first sealing plate around a periphery of the first channel; and
a cannula fluidly coupled to the first channel, the cannula adapted to be inserted into the anterior chamber of an eye and to provide a heated fluid for removal of a cataractous lens; and
a hand piece segment comprising:
a second sealing plate located on a top surface of the hand piece segment, the second sealing plate having a second channel, the second channel for receiving a fluid;
a receiving portion extending from the second sealing plate around a periphery of the second channel, the receiving portion movable in an axial direction along the second channel, the receiving portion biased outward from a surface of the second sealing plate;
wherein when the ring portion is pressed against the receiving portion, the receiving portion provides a sealing force against the ring portion such that the first and second channels provide a continuous leak-resistant path for a fluid.

16. The fluidic coupling of claim 15 further comprising:
a spring located adjacent to the receiving portion, the spring for biasing the receiving portion toward the ring portion and for providing the sealing force.

17. The fluidic coupling of claim 15 further comprising:
a spring washer located adjacent to the receiving portion, the spring washer for biasing the receiving portion toward the ring portion and for providing the sealing force.

18. The fluidic coupling of claim 15 further comprising:
a third channel located on the first sealing plate;
a fourth channel located on the second sealing plate; and
a gasket located between the first and second sealing plates.

19. The fluidic coupling of claim 18 wherein when the ring portion is pressed against the receiving portion, the third and fourth channels provide a continuous path for irrigation.

20. The fluidic coupling of claim 18 wherein when the ring portion is pressed against the receiving portion, the third and fourth channels provide a continuous path for aspiration.
